(19) United States
(12) Patent Application Publication
(10) Pub. No.: US 2003/0045849 A1
(43) Pub. Date: Mar. 6, 2003

(54) Medical laminate with viral barrier

(75) Inventors: John Steffen, Sugar Hill, GA (US); Keith Wilbourn, Matthews, NC (US)

Correspondence Address: WOOD, PHILLIPS, KATZ, CLARK & MORTIMER, 500 W. MADISON STREET, SUITE 3800, CHICAGO, IL, 60661, US

(73) Assignee: Polymer Group, Inc.

(21) Appl. No.: 10/136,678

(22) Filed: May 1, 2002

Related U.S. Application Data
(60) Provisional application No. 60/288,188, filed on May 2, 2001.

(51) Int. Cl. A61F 13/15; B32B 27/00

(52) U.S. Cl. 604/370; 442/394

(57) ABSTRACT

The present invention is directed to a nonwoven fabric construct in the form of a laminate material suitable for medical applications. The laminate includes a nonwoven fabric layer, and first and second polymeric film layers, with the resultant material exhibiting superior viral protection in accordance with established testing procedures, ASTM F1671. In accordance with the present invention, the first and second polymeric film layers are co-extruded and adhered to one surface of the nonwoven fabric layer, with the second polymeric film layer being disposed between the first polymeric film layer and the nonwoven fabric layer. Notably, the first polymeric film layer comprises a blend of between about 0 to 100% low density polyethylene, and between about 0 to 100% linear low density polyethylene.

### TECHNICAL FIELD

The present invention relates generally to laminate materials formed from nonwoven fabrics and polymeric films, and more particularly to a laminate material comprising a nonwoven fabric, and first and second polymeric film layers, with the resultant material providing a highly effective viral barrier suitable for medical applications.

### BACKGROUND OF THE INVENTION

Nonwoven fabric constructs are used in a very wide variety of applications in which the engineered qualities of such materials can be advantageously employed. Nonwoven fabric webs may be formed from fibrous material in the form of natural or synthetic fibers, or substantially continuous filaments, with the materials from which such fabrics are formed, and the nature of the fabrication process, determining the physical characteristics of the resultant fabric. Nonwoven fabric constructs may include plural or composite fabric layers, and may also include composite structures formed from laminations of nonwoven fabrics and polymeric films.

Nonwoven fabric constructs have proven to be particularly suitable for a variety of medical applications since they permit cost-effective, disposable use. Use of such materials for medical gowns and the like has become increasingly widespread, since the physical properties and characteristics of the nonwoven fabric constructs can be selected as may be required for specific medical applications. U.S. Pat. No. 5,748,167, hereby incorporated by reference, discloses a nonwoven fabric laminate construct stated as being useful for protective apparel in view of its barrier properties and durability; U.S. Pat. No. 5,981,038, also hereby incorporated by reference, discloses a micro-porous membrane, which can be laminated to a fabric, which is stated as preventing transmission of viral pathogens.

For some medical applications, it is important that a nonwoven fabric construct function as a viral barrier, so that clothing formed from such a material provides the necessary protection against blood, body fluids, and other potentially infectious materials. While nonwoven fabric materials in the form of laminates of nonwoven fabrics and polymeric films have been used in the past, such materials have typically included a single polymeric film layer. However, testing has shown that such constructs do not provide the desired level of viral protection in a cost-effective fashion.

The present nonwoven fabric construct is intended to provide improved viral protection, thereby facilitating use of the material for medical applications, with the present material lending itself to cost-effective, disposable use.

### SUMMARY OF THE INVENTION

The present invention is directed to a nonwoven fabric construct in the form of a laminate material suitable for medical applications. The laminate includes a nonwoven fabric layer, and first and second polymeric film layers, with the resultant material exhibiting superior viral protection in accordance with established testing procedures, ASTM F1671.

In accordance with the present invention, the first and second polymeric film layers are co-extruded and adhered to one surface of the nonwoven fabric layer, with the second polymeric film layer being disposed between the first polymeric film layer and the nonwoven fabric layer. Notably, the first polymeric film layer comprises a blend of between about 0 to 100% low density polyethylene, and between about 0 to 100% linear low density polyethylene.

In the preferred form, the blended materials of the first polymeric film layer are provided in a weight ratio of the low density polyethylene to the linear low density polyethylene from about 75:25 to about 65:35. The first polymeric film layer may also comprise up to about 10%, by weight, of a pigment. In a presently preferred formulation, the weight ratio of the low density polyethylene to the linear low density polyethylene is about 70:30, with the pigment comprising about 4%, by weight, of the first polymeric film layer.

The second polymeric film layer provides adhesion between the first polymeric film layer and the nonwoven fabric layer, and in a current embodiment, comprises ethylene methyl acrylate. The second polymeric film layer may also comprise up to about 10% of a pigment. The second polymeric film layer may be subjected to ozone treatment for adhesion enhancement, with adhesion enhanced by subjecting the nonwoven fabric layer to corona discharge treatment.

The first polymeric film layer and the second polymeric film layer have a weight ratio from about 75:25 to 55:45, more preferably about 70:30 to 60:40, and most preferably 67:33 (2 to 1).

While the nonwoven fabric layer of the present laminate may be provided in many different forms, an adhesive-bonded, carded polyester fiber web is presently preferred for cost-effectiveness. In a current embodiment, a polyester web having a basis weight of 21 g/m² was employed, with the first and second polymeric film layers having a combined basis weight of 31 g/m².

Other features and advantages of the present invention will become readily apparent from the following detailed description, and the appended claims.

### DETAILED DESCRIPTION

While the present invention is susceptible of embodiment in various forms, there will hereinafter be described, a presently preferred embodiment, with the understanding that the present disclosure is to be considered as an exemplification of the invention, and is not intended to limit the invention to the specific embodiment illustrated.

The present invention is directed to a nonwoven fabric construct provided in the form of a laminate material formed by extrusion coating of polymeric film layers on a nonwoven fabric base layer. As will be described, the present laminate comprises an adhesive-bonded polyester fiber nonwoven fabric, and olefin film, with the product exhibiting sufficient viral protection to permit use for those medical applications where this type of protection is required. ASTM 1671, hereby incorporated by reference, specifies test protocols for testing materials for such medical applications.

In an exemplary form, the present laminate material comprises a 21 μm² adhesive bonded, polyester carded web laminated with 31 g/m² of co-extruded olefin film. The olefin film is provided in the form of first and second polymeric film layers. The first polymeric film layer comprises a blend of between about 0 to 100% low density polyethylene (LDPE), and between about 0 to 100% linear low density polyethylene (LLDPE). The preferred LDPE to LLDPE ratio is from about 75:25 to 65:35, with presently preferred compositions having a weight ratio of about 70:30. Pigments may be added to the first polymeric layer, up to 10% by weight, with the LDPE to LLDPE ratios adjusted to the above-noted range. Presently preferred pigment addition is 4% by weight. By the above-described composition, the first polymeric layer provides desired strength and barrier properties for the present laminate material.

The second polymeric layer provides adhesion strength between the first polymeric film layer and the associated nonwoven fabric layer. In a presently preferred embodiment, the second polymeric film layer comprises 100% ethylene methyl acrylate (EMA). Like the first polymeric film layer, the second polymeric film layer may also include a pigment, up to about 10%, with about 4%, by weight of pigment, being presently preferred.

The first polymeric film layer (LDPE) and the second polymeric film layer (LLDPE) have a weight ratio of about 75:25 to 55:45, more preferably about 70:30 to 60:40, with a 67:33 (2 to 1) ratio being presently most preferred. In a presently preferred embodiment, the nonwoven fabric base layer or substrate comprises an adhesive bonded, polyester carded web. In a current embodiment, the polyester carded web was formed with a basis weight of 21 g/m², with the combined first and second polymeric film layers having a total basis weight of 31 g/m² bonded to the nonwoven fabric layer. It is within the purview of the present invention that a variety of nonwoven fabric layers can be used in combination with the first and second polymeric film layers, including spunbond, melt-blown, and carded fabric constructs, with fibers or filaments formed from polypropylene, polyethylene, rayon, or cotton. A variety of different fibrous substrates can be employed since the first and second polymeric film layers act together to provide the desired barrier protection for the present laminate material. Adhesion of the polymeric film layers to the nonwoven fabric layer can be enhanced by subjecting the fabric layer to corona discharge treatment.

The polymeric film layers are applied to the nonwoven fabric substrate using a dual manifold cast film die. However, any die with combining block technology can also be used. The polymeric film layers and nonwoven fabric substrate are combined in a nip shortly after the extrudate leaves the die. The two rolls used in the nip are a matte finished seal roll, and a rubber covered steel roll. The rolls are both cooled to between 35° F. and 80° F.

Viral barrier testing is conducted in accordance with ASTM F 1671. Experience has shown that when extrusion coating is employed for providing a single layer on an associated nonwoven fabric web, the single polymeric layer exhibits pinholes which compromise the various properties. When a nonwoven fabric has a bi-layer material extruded on to it, the pinholes exhibited in a single layer are likely overlaid with a stiffer polyethylene layer. The probability of two pinholes directly on top of one another is very small, and statistically insignificant as a pathway for viral penetration.

The laminate material embodying the principles of the present invention has been found to pass the ASTM F1671 viral barrier testing. The use of the inner (second) polymeric film layer improves adhesion between the film and nonwoven layers for the construct. Notably, the present laminate material can be formed such that the film layers are coated on either side of the nonwoven fabric layer.

A notable feature of the present laminate material is its use of existing materials, which have been validated for current applications. The total basis weight of the film layers is similar to existing products, as is the total composition of the film, and the nonwoven fabric which is used.

The following describes testing conducted in connection with development of the present laminate material.

REFERENCES: ASTM F 1671-97b. Standard Test Method For Resistance of Materials Used in Protective Clothing to Penetration by Blood-Borne Pathogens Using Phi-X174 Bacteriophase Penetration as a Test System. American Society For Testing and Materials, West Conshohocken, Pa.

NFPA 1999. 1997. Standard on Protective Clothing for Emergency Medical Operations. National Fire Protection Association, Quincy, Mass.

Calendar, Richard. The Bacteriophages. Vol. 2.

This describes details and results for the microbiological viral penetration testing of protective clothing materials, which are to be used to protect against blood borne pathogen hazards. The test procedure was designed to comply with the ASTM test method F1671-97b Standard Test Method for Resistance of Materials Used in Protective Clothing to Penetration by Blood-Borne Pathogens Using Viral Penetration as a Test System. Formerly, this test method was designated as ASTM ES22-92. The test device used in this procedure was the ASTM F903 Chemical Penetration Test.

The blood-borne pathogens of major concern are the hepatitis B virus (HBV), hepatitis C virus (HCV), and human immunodeficiency virus (HIV). HBV is an enveloped, spherical, 42-47 nm virus. HVC is a nonenveloped, icosahedral, 27-30 nm virus. HIV is an enveloped, spherical 80-110 nm virus. The blood serum concentrations of these three blood-borne pathogens ranges from less than 100 to more than 100 million IU/mL (infectious unit per milliliter). The φX174 bacteriophage is one of the smallest known viruses. It is a nonenveloped, icosahedral, 25-27 nm virus. The φX174 bacteriophage challenge suspension was maintained at a concentration of at least 1.0×10⁸ PFU/mL (plaque forming units/mL).

The protective clothing materials tested are intended to provide protection against blood, body fluids, and other potentially infectious materials. The surface tension range for blood and body fluids is approximately 42-60 dynes/cm. In order to simulate the wetting characteristics of blood and body fluids the surface tension of the φX174 bacteriophage suspension was adjusted to approximate the lower end of this surface tension range (40-44 dynes/cm).

The choice of a microbiological model to evaluate the effectiveness of the blood-borne pathogen barrier properties of protective clothing materials is important. There are problems associated with utilizing the actual blood-borne pathogens at test organisms. HBV and HCV cannot be grown in the laboratory. HIV represents a significant safety and liability consideration due to its high infectivity potential and requirements for extreme and expensive precautions.

A model for the blood-borne pathogens was researched. The ideal properties of a surrogate would include small size, spherical or polyhedral (round) morphology, environmental stability, low or non-human infectivity, high assay sensitivity, rapid growth, and high titer. The φX174 bacteriophage was selected as the most appropriate surrogate for the blood-borne pathogens mentioned because it satisfies all of these criteria. The φX174 bacteriophage is a nonenveloped, 25-27 nm virus (similar to HCV, the smallest pathogen mentioned), with icosahedral or nearly spherical morphology similar to all three viral pathogens mentioned. It has excellent environmental stability, is noninfectious to humans, has a limit of detection which approaches a single virus particle, grows rapidly, and can be cultivated to reach high titers similar to HBV (the most concentrated pathogen mentioned).

Animal virus surrogates are not used as they require specialized cell culture and enzyme assay techniques. In addition, the stability of most of the animal viruses is less than desirable and plating efficiency is low or unknown.

Despite the variety of viral coats or surfaces (i.e., lipophilic, hydrophilic, etc.), they generally perform similarly in barrier or penetration tests. This is because viruses adopt the charge of the liquid in which they are suspended and are more affected by the liquid vehicle than by their own physical or chemical properties.

It is also important to note that while blood may seem appropriate as the test vehicle, it is actually a poor choice. Many viruses adsorb to blood cells. Red blood cells are about 7-10 microns in diameter and can actually plug pores. Since many other body fluids can be infectious, it is more severe to use a body fluid simulant (surfactant containing, particulate-free suspending liquid) such as that described in this procedure.

Analysis of materials for compatibility with the test organisms is important due to the possibility that the test material could contain some substances which may be inhibitory to the virus or to the host bacterium. Recovery of the virus may also be lowered when testing materials which are more absorbent due to the possibility that the virus may remain bound to the material so that it is not picked up in the assay fluid.

TEST SPECIMEN PREPARATION: Test specimens, formed in accordance with the presently preferred embodiment of the present invention (corona treatment of fabric, no ozone treatment of second film layer), measuring approximately 75×75 mm were cut at random from the smooth portions of the test material. The Samples were conditioned for a minimum of 24 hours at 21±5° C. and 30 to 80% relative humidity.

COMPATIBILITY TESTING: Compatibility testing was performed by placing a 2.0 microliter aliquot of a φX174 suspension containing a total of 900-1200 PFU near the center of the test sample after it had been clamped into the penetration test cell. After 60 minutes, the surface was rinsed with a sterile assay medium and then assayed for the presence of the φX174 bacteriophage.

To calculate the ratio of the control assay titer to the test material assay titer, the following equation was used:

$$\text{ratio} = \frac{\text{control assay titer} ( PFU \text{/} mL )}{\text{test material assay titer} ( PFU \text{/} mL )}$$

The titer of the φX174 bacteriophage challenge suspension used for the test was 2 (+/−1)×108 PFU/mL times the ratio calculated. The compatibility ratio was 1.8, the range of the prechallenge titer should be 2.6×10⁸ PFU/mL to 4.6×10⁸ PFU/mL.

TEST PROCEDURE: The test samples were loaded into the test cell with the film side of the test specimen toward the viral challenge. The test cell bolts were torqued to 13.6 Newton meters (120 inch pounds) in a criss-cross technique. Test samples were challenged with approximately 60 mL of a φX174 bacteriophage suspension for 5 minutes at atmospheric pressure, 1 minute at 2.0 psig (13.8 kPa), and 54 minutes at atmospheric pressure or until liquid penetration was observed. A retaining screen was not used in accordance with procedure A as outlined in ASTM F-1671b. At the conclusion of the test procedure, the bacteriophage suspension was drained from the test cell and collected to determine the post φX174 bacteriophage concentration. The observed side of the test sample was rinsed with 5 mL of a sterile assay medium and then recovered from the surface of the sample with a sterile pipette. The collected sample fluid was assayed using 0.5 mL (in duplicate) for the presence of the φX174 bacteriophage. The surface tension of the challenge suspension and the assay medium was adjusted to approximately 40-44 dynes/cm using surfactant-type TWEEN 80 (a registered trademark of ICI Americas Inc., of Delaware), at a final concentration of approximately 0.01% by volume.

Following testing, the samples were allowed to dry and the thickness of each specimen was determined using a thickness dial gauge.

TEST CONTROLS: A negative control sample was included in the study to show that a negative result could be obtained when challenged with the φX174 bacteriophage. The negative control material used was a sterile 2 mil polyethylene film that has consistently not allowed φX174 penetration when tested according to this procedure.

A positive control was also included in the study to show that the φX174 bacteriophage could be recovered using the assay procedure described. The positive control sample consisted of a 0.04 micron porous membrane that has consistently allowed φX174 penetration to occur.

Because the test samples were not sterilized prior to testing, a control blank was included in the testing program. The blank was a sample cut from the test material and it was challenged with sterile nutrient broth with 0.01% TWEEN® 80. The blank was used to ensure that the test material, as received, did not contain any background contamination which may have adversely affected the test results.

Fallout plates were used during the testing procedure. The fallout plates consisted of bottom agar overlaid with top agar and Escherichia coli, serotype C. The fallout plates were strategically placed on the work bench area to determine the background counts (if any) from airborne contamination.

RESULTS: Refer to Table 1 for a summary of the test results.

The results of the negative control sample (2 mil polyethylene) showed no φX174 penetration under the test conditions indicating proper aseptic technique was demonstrated. The positive control sample (0.04 micron porous membrane) showed significant φX174 penetration which demonstrates that assay procedure was effective in recovering the φX174 challenge using this test procedure. Refer to Table 2 for a summary of the results for the test controls.

The results of the fallout plates indicate the testing environment was acceptable.

TABLE 1
Viral Penetration Results
ASTM Method F1671-97b
Exposure Procedure Used: A
Sample ID: #1, #3, #5, and #7

| | SPECIMEN | PRECHALLENGE | POSTCHALLENGE | ASSAY | | |
|---|---|---|---|---|---|---|
| SAMPLE | THICKNESS | CONCENTRATION | CONCENTRATION | TITER | VISUAL | TEST |
| ID | (mm) | (PFU/mL) | (PFU/mL) | (PFU/mL) | PENETRATION | RESULT |
| 1-1 | 0.15 | 2.9 × 10⁸ | 2.5 × 10⁸ | <1* | None Seen | Pass |
| 1-2 | 0.13 | 2.9 × 10⁸ | 2.5 × 10⁸ | <1* | None Seen | Pass |
| 1-3 | 0.12 | 2.9 × 10⁸ | 2.5 × 10⁸ | <1* | None Seen | Pass |
| 3-1 | 0.13 | 2.9 × 10⁸ | 2.5 × 10⁸ | <1* | None Seen | Pass |
| 3-2 | 0.13 | 2.9 × 10⁸ | 2.5 × 10⁸ | <1* | None Seen | Pass |
| 3-3 | 0.15 | 2.9 × 10⁸ | 2.5 × 10⁸ | <1* | None Seen | Pass |
| 5-1 | 0.13 | 2.9 × 10⁸ | 2.5 × 10⁸ | <1* | None Seen | Pass |
| 5-2 | 0.12 | 2.9 × 10⁸ | 2.5 × 10⁸ | <1* | None Seen | Pass |
| 5-3 | 0.13 | 2.9 × 10⁸ | 2.5 × 10⁸ | <1* | None Seen | Pass |
| 7-1 | 0.12 | 2.9 × 10⁸ | 2.5 × 10⁸ | <1* | None Seen | Pass |
| 7-2 | 0.14 | 2.9 × 10⁸ | 2.5 × 10⁸ | <1* | None Seen | Pass |
| 7-3 | 0.12 | 2.9 × 10⁸ | 2.5 × 10⁸ | <1* | None Seen | Pass |

*A value of <1 PFU/mL is reported for assay plates showing no plaques.

TABLE 2
Viral Penetration Results
ASTM Method F1671-97b
Exposure Procedure Used: A
Test Controls

| | PRECHALLENGE | POSTCHALLENGE | ASSAY | | |
|---|---|---|---|---|---|
| CONTROL | CONCENTRATION | CONCENTRATION | TITER | VISUAL | TEST |
| SPECIMENS | (PFU/mL) | (PFU/mL) | (PFU/mL) | PENETRATION | RESULTS |
| Negative | 2.9 × 10⁸ | 2.5 × 10⁸ | <1^{a} | None Seen | Pass |
| Control | | | | | |
| Positive | 2.9 × 10⁸ | 2.5 × 10⁸ | 1.1 × 10² | Yes | Fail |
| Control | | | | | |
| Blank | N/A^{b} | N/A^{b} | 1^{a} | None Seen | Pass |

^{a}A value of <1 PFU/mL is reported for assay plates showing no plaques.
^{b}The blank was challenged with sterile nutrient broth with 0.01% Tween ® 80.

What is claimed is:

1. A laminate material suitable for material applications, comprising:
a nonwoven fabric layer; and
first and second polymeric film layers, said second polymeric film layer being imposed between said first polymeric film layer and said nonwoven fabric layer;
said first polymeric film layer comprising a lend of between about 0 to 100% low density polyethylene, and between about 0 to 100% linear low density polyethylene,
said second polymeric layer providing adhesion between said first polymeric layer and said nonwoven fabric layer.

2. A laminate material in accordance with claim 1, wherein:
said second polymeric layer comprises ethylene methyl acrylate.

3. A laminate material in accordance with claim 2, wherein:
said second polymeric layer further comprises up to 10% of a pigment.

4. A laminate material in accordance with claim 2, wherein:
said second polymeric layer is subjected to ozone treatment for adhesion enhancement.

5. A laminate material in accordance with claim 1, wherein:
said first polymeric layer has a weight ratio of said low density polyethylene to said linear low density polyethylene from about 75:25 to about 65:35.

6. A laminate material in accordance with claim 5, wherein:
said first polymeric layer comprises up to about 10%, by weight, of a pigment.

7. A laminate material in accordance with claim 6, wherein:
said weight ratio of said low density polyethylene to said linear low density polyethylene is about 70:30, and said pigment comprises about 4%, by weight, of said first polymeric layer.

8. A laminate material in accordance with claim 1, wherein:
said nonwoven fabric layer comprises adhesive bonded, carded polyester fibers.

9. A laminate material in accordance with claim 8, wherein:
said nonwoven fabric layer is subjected to corona discharge treatment for adhesion enhancement.

10. A laminate material in accordance with claim 1, wherein
said first polymeric layer and said second polymeric layer have a weight ratio of about 75:25 to 55:45.

11. A method of making a laminate material, comprising the steps of:
providing a nonwoven fabric layer; and
co-extruding first and second polymeric film layers onto said nonwoven fabric layer so that said second polymeric film layer is disposed between said first polymeric film layer and said nonwoven fabric for adhesion therebetween;
said first polymeric film layer comprising a blend of between about 0 to 100% low density polyethylene, and between about 0 to 100% linear low density polyethylene.

12. A method of making a laminate material in accordance with claim 11, wherein:
said second polymeric film layer comprises ethylene methyl acrylate.

13. A method of making a laminate material in accordance with claim 12, including:
subjecting said second polymeric film layer to at least one of corona discharge treatment and ozone treatment for adhesion enhancement.

14. A method of making a laminate material in accordance with claim 11, including:
subjecting said nonwoven fabric layer to corona discharge treatment for adhesion enhancement.

15. A method of making a laminate material in accordance with claim 11, wherein:
said first polymeric layer has a weight ratio of said low density polyethylene to said linear low density polyethylene from about 75:25 to about 65:35.

16. A method of making a laminate material in accordance with claim 12, wherein:
said first polymeric layer comprises up to about 10%, by weight, of a pigment.

17. A method of making a laminate material in accordance with claim 11, wherein:
said first polymeric layer and said second polymeric layer have a weight ratio of about 75:25 to 55:45.

18. A laminate material suitable for medical applications, comprising:
a nonwoven fabric layer; and
first and second polymeric film layers, said second polymeric film layer being imposed between said first polymeric film layer and said nonwoven fabric layer;
said first polymeric film layer comprising a blend of about 0 to 100% low density polyethylene, and between about 0 to 100% linear low density polyethylene, said second polymeric layer providing adhesion between said first polymeric layer and said nonwoven fabric layer;
the resulting laminate material exhibiting a viral barrier performance that meets or exceeds ASTM F1671.
